(19) United States
(12) Patent Application Publication
(10) Pub. No.: US 2015/0104090 A1
(43) Pub. Date: Apr. 16, 2015

(54) MODIFICATION OF A HOLLOW ORGAN REPRESENTATION

(71) Applicant: Siemens Aktiengesellschaft, Munich (DE)

(72) Inventors: Christian HOPFGARTNER, Fuerth (DE); Jan KRETSCHMER, Nuernberg (DE); Max SCHÖBINGER, Hirschaid (DE)

(21) Appl. No.: 14/496,222

(22) Filed: Sep. 25, 2014

(30) Foreign Application Priority Data
Oct. 11, 2013 (DE) 102013220539.2

(51) Int. Cl. A61B 6/00; G06T 7/00; A61B 8/08; A61B 6/03; A61B 5/055

(52) U.S. Cl. CPC A61B 6/5205; A61B 6/032; A61B 6/037; A61B 5/055; A61B 6/504; A61B 8/0891; A61B 8/5207; G06T 7/0012; G06T 2207/10081; G06T 2207/10088; G06T 2207/10104; G06T 2207/30104
USPC 382/131

(57) ABSTRACT

A method and a system are disclosed for interactively creating and/or modifying a hollow organ representation on the basis of medical-technical image data of a hollow organ. An embodiment of the method includes providing the medical-technical image data together with a hollow organ course line representing the course of the hollow organ; providing a plurality of contour representations of a contour of the hollow organ representation along the hollow organ course line; receiving a command input for the input and/or modification of a selected contour representation and/or of the hollow organ course line; locally modifying (H) the contour of the hollow organ representation on the basis of the command input, taking into consideration a number of contour representations adjacent to the selected contour representation on at least one side along the hollow organ course line, using an automatic interpolating sweep algorithm.

### PRIORITY STATEMENT

The present application hereby claims priority under 35 U.S.C. §119 to German patent application number DE 102013 220539.2 filed Oct. 11, 2013, the entire contents of which are hereby incorporated herein by reference.

### FIELD

At least one embodiment of the present invention generally relates to a method for interactively creating and/or modifying a hollow organ representation on the basis of medical-technical image data of a hollow organ. It also generally relates to a creation and/or modification system for interactively creating and/or modifying a hollow organ representation and/or a hollow organ course line of a hollow organ representation on the basis of medical-technical image data of a hollow organ of an organism. In particular, at least one embodiment of the invention is applicable in the field of hollow organs which are or comprise blood vessels.

### BACKGROUND

Patient-specific models of vascular structures provide an important basis for numerous clinical applications. Owing to their complex morphology and their life-critical function, (blood) vessels have a particular significance when evaluating risks of various surgical approach strategies.

In this context, the simulation of blood flow (computational hemodynamics) is one example of an important new field which is closely linked to medical-technical imaging. The underlying idea is to avoid invasive measuring methods and the associated patient risks by instead using blood vessel segmenting from medical-technical image data in order to simulate quantifiable patient-specific blood flows. These simulations can be used to calculate and visualize e.g. degrees of severe wall stress of the vessel or so-called fractional flow reserve (FFR) statistics, or flow patterns and models within an aneurysm.

In order for the performance of such simulations to be properly meaningful, accurate vascular models are of the utmost importance. In pathological cases in particular, it is not possible to maintain the simplistic assumption that blood vessels have a mainly circular cross-sectional area. In order to represent stenoses or aneurysms precisely, detailed and meaningful modeling and segmenting techniques are therefore required.

Automatic vessel segmenting is already an established area in the field of medical-technical imaging, and a great many methods and procedures have been developed in this area. However, none of these methods guarantees perfect segmenting for all conceivable scenarios.

One option for vessel segmenting is the use of interpolating sweep algorithms such as those disclosed in Ryan Schmidt/Brian Wyvill: “Generalized Sweep Templates for Implicit Modeling”, in: GRAPHITE '05 Proceedings of the 3rd International Conference on Computer Graphics and Interactive Techniques in Australia and South East Asia, pages 187-196, New York, 2005, doi: 10.1145/1101389.1101428. The sweeping method can be used to model the entire vascular tree on the basis of a vessel course line which has been determined, for example. However, this method requires considerable computational resources and is associated with uncertainties like all other methods.

In particular, pathological cases present significant challenges for vessel segmenting algorithms. At present, this means that automatically generated segmentation results must be examined “manually”, i.e. visually by a specialist, before computational hemodynamics can be determined. If errors or problems occur in the computational hemodynamics, one approach is to repeat one or more segmenting actions using a modified parameter set, which is expected to yield better segmentation results. Another approach is to export data (i.e. image data) to generic modeling applications or tools, in which proficient experts make valid corrections. However, such a workflow is relatively difficult to apply in the everyday clinical routine since it requires highly developed geometric modeling knowledge and complex tools, and is too time-intensive.

An interactive correction, i.e. a modification or possibly an interactive creation by experts of vascular models which have been segmented on the basis of medical-technical image data is often desirable in order to increase as far as possible the desired segmentation accuracy and hence the meaningfulness of the segmented hollow organ representation in the image data.

### SUMMARY

At least one embodiment of the present invention is to directed to an improved creation or modification of a hollow organ representation or individual parameters (parameter sets) thereof according to the result.

A method and a creation and/or modification system are disclosed.

In this case, an inventive method of the type cited in the introduction comprises steps as follows:

a) providing the medical-technical image data together with a hollow organ course line representing the course of the hollow organ,
b) providing a plurality of contour representations of a contour of the hollow organ representation along the hollow organ course line,
c) receiving a command input for the input and/or modification of a selected contour representation and/or of the hollow organ course line,
d) locally modifying the contour of the hollow organ representation on the basis of the command input, taking into consideration a predefined number of contour representations which are adjacent to the selected contour representation on at least one side along the hollow organ course line, using an automatic interpolating sweep algorithm.

At least one embodiment of the invention therefore also comprises a computer program product which can be loaded directly into a processor of a programmable creation and/or modification system, having program code segments for executing all the steps of a method according to at least one embodiment of invention (also in accordance with the aspects described below) when the program product is executed on the creation and/or modification system.

At least one embodiment of the invention further comprises a medical-technical recording system comprising a recording unit (i.e. an acquisition unit) and a creation and/or modification system according to at least one embodiment of the invention (also in accordance with the aspects described below).

A creation and/or modification system according to at least one embodiment of the invention, for interactively creating and/or modifying a hollow organ representation on the basis of medical-technical image data of a hollow organ of an organism, comprises:

a) a first provision unit which, during operation, provides the medical-technical image data together with a hollow organ course line representing (at least approximately) the course of the hollow organ,
b) a second provision unit which, during operation, provides a plurality of contour representations of a contour of the hollow organ representation along the hollow organ course line,
c) a receive interface for receiving a command input for the input and/or modification of a selected contour representation and/or of the hollow organ course line,
d) a modification unit which is designed to locally modify the contour of the hollow organ representation on the basis of the command input, taking into consideration a predefined number of contour representations which are adjacent to the selected contour representation on at least one side along the hollow organ course line, using an automatic interpolating sweep algorithm.

The first further aspect relates to a method for preferably automatic modification of a hollow organ course line of a hollow organ representation (wherein the hollow organ again comprises specifically a blood vessel or a blood vessel tree) on the basis of medical-technical image data of a hollow organ (of an organism), in particular in the context of the method according to at least one embodiment of the invention as described above for interactively creating and/or modifying a hollow organ representation. The method of at least one embodiment comprises at least the following steps:

i) providing the medical-technical image data together with the hollow organ course line representing (again at least approximately) the course of the hollow organ,
ii) providing a plurality of contour representations of a contour of the hollow organ representation along the hollow organ course line,
iii) determining a deviation of the hollow organ course line from contour centerpoints of two adjacent contour representations,
iv) adapting the hollow organ course line with the aid of the two contour centerpoints if a predetermined maximal tolerated deviation of the hollow organ course line from the two contour centerpoints is exceeded, and
v) optionally: locally modifying the contour of the hollow organ representation on the basis of the adapted hollow organ course line.

### BRIEF DESCRIPTION OF THE DRAWINGS

The invention is explained again in greater detail below with reference to the appended figures and with reference to example embodiments. In this case, identical components are denoted by identical reference numerals in the various figures, in which:

FIG. 1 shows a schematic sequence diagram including example embodiments of methods according to an embodiment of the invention,

FIG. 2 shows a schematic block diagram of an example embodiment of a method according to the invention for interactively creating and/or modifying a hollow organ representation,

FIG. 3 shows a schematic block diagram of an example embodiment of a method according to the invention for modifying a hollow organ course line of a hollow organ representation,

FIG. 4 shows a schematic block diagram of an example embodiment of a method according to the invention for modifying a contour representation of a hollow organ representation,

FIG. 5 shows a schematic block diagram of an example embodiment of a creation and/or modification system according to the invention,

FIG. 6 shows an example illustration of the conversion of a contour representation in the context of an example embodiment of the method according to the invention for interactively creating and/or modifying a hollow organ representation,

FIG. 7 shows an example illustration of performing an example embodiment of a sweeping method of the type which can be used in the context of the invention,

FIG. 8 shows a perspective illustration of results of different example embodiments of sweeping methods which can be used in the context of the invention,

FIG. 9 shows a perspective illustration of a hollow organ representation of the type which can be generated or modified according to an example embodiment of the invention,

FIG. 10 shows two perspective illustrations of one and the same hollow organ representation during the performance of an example embodiment of the method according to the invention for modifying a hollow organ course line of a hollow organ representation,

FIG. 11 shows a perspective internal view of a hollow organ representation of the type which can be generated or modified according to an example embodiment of the invention,

FIG. 12 shows two perspective illustrations of hollow organ representations before and after user intervention in the context of an example embodiment of the invention,

FIG. 13 shows four perspective illustrations of one and the same hollow organ representation during the performance of an example embodiment of the method according to the invention for modifying a contour representation of the hollow organ representation.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

A method and a creation and/or modification system are disclosed.

In this case, an inventive method of the type cited in the introduction comprises steps as follows:

a) providing the medical-technical image data together with a hollow organ course line representing the course of the hollow organ,
b) providing a plurality of contour representations of a contour of the hollow organ representation along the hollow organ course line,
c) receiving a command input for the input and/or modification of a selected contour representation and/or of the hollow organ course line,
d) locally modifying the contour of the hollow organ representation on the basis of the command input, taking into consideration a predefined number of contour representations which are adjacent to the selected contour representation on at least one side along the hollow organ course line, using an automatic interpolating sweep algorithm.

In particular, the hollow organ representation is preferably a representation of a blood vessel or blood vessel tree in this case, and therefore in this specific preferred case reference may also be made to a blood vessel representation or a blood vessel tree representation. The hollow organ is preferably a hollow organ of an organism, in particular a human being. The organism is preferably a living organism.

The provision of the medical-technical image data and/or the corresponding hollow organ course line in step a) may include its creation, i.e. the medical-technical image data may be created in step a) by way of image acquisition using a medical-technical tomography system. Such tomography systems include e.g. computed tomography systems (CT), magnetic resonance tomography systems (MR), angiographs, X-ray apparatuses, ultrasound devices, single proton emission computed tomography systems (SPECT), positron emission tomography systems (PET), etc. The hollow organ course line may be a so-called centerline in particular, i.e. a central line of the hollow organ. Said line represents at least approximately the course of the hollow organ concerned. Like the medical-technical image data, it may also be generated in the context of step a), e.g. manually by a user and/or automatically by a determining algorithm. Semi-automatic determination is also possible, i.e. determination which is automatic but assisted by a user.

The medical-technical image data and/or the hollow organ course line may also be procured from a database in step a). This means that the medical-technical image data or the hollow organ course line was acquired or determined previously, is present in the database and is simply procured (i.e. retrieved) via an input interface in step a).

The provision of the contour representations in step b) may likewise comprise a creation thereof and/or procurement (i.e. retrieval) from a database, said database possibly being the same one that was optionally used in step a) or a further (independent) database. The contour representations may comprise virtual contour representations, i.e. they may have been determined on the basis of a computer-based algorithm.

The optional modification of a specific, i.e. selected, contour representation may also comprise a deletion thereof, as in the case of a complete new input by a user in the context of the interactive process. This means that the selected contour representation may be selected either from those provided in step b) (which is preferred) or newly generated.

In step d), the contour of the hollow organ representation is modified locally. “Locally” means here that instead of the whole hollow organ representation, merely a selected part thereof is newly created, i.e. calculated, specifically that part which is identified by the predefined number of adjacent contour representations. These adjacent contour representations lie on at least one side of the selected contour representation along the hollow organ course line, and most preferably on both sides of the selected contour representation. In this preferred variant, the number is preferably selected such that the same number of adjacent contour representations is taken into consideration on both sides of the selected contour representation and/or an essentially identical distance along the hollow organ course line is taken into consideration.

According to at least one embodiment of the invention, provision is therefore made for a local modification of the hollow organ representation using a sweep algorithm, instead of a complete recalculation as is generally known. This procedure has the clear advantage that the computing time and computing effort required for the interactive creation of the hollow organ representation can be significantly reduced. Specifically, if a user input relating to a selected contour representation is made according to the prior art, several seconds are required before the result of the complete recalculation is displayed to the user. In the context of a workflow where the user routinely performs not just one but often a plurality of such inputs, this acceleration therefore represents a significant advantage and moreover assists the intuitive actions of the user, who can see the new result in the form of the new hollow organ representation almost instantly following the user input. Furthermore, this exclusively local adaptation of the hollow organ representation also serves to prevent new errors, since the sweep algorithm does not simply pass along the whole hollow organ course line on the basis of the input, but is also halted again by the predetermined number of adjacent contour representations under consideration, thereby avoiding any successive distortions.

A creation and/or modification system according to at least one embodiment of the invention, for interactively creating and/or modifying a hollow organ representation on the basis of medical-technical image data of a hollow organ of an organism, comprises:

a) a first provision unit which, during operation, provides the medical-technical image data together with a hollow organ course line representing (at least approximately) the course of the hollow organ,
b) a second provision unit which, during operation, provides a plurality of contour representations of a contour of the hollow organ representation along the hollow organ course line,
c) a receive interface for receiving a command input for the input and/or modification of a selected contour representation and/or of the hollow organ course line,
d) a modification unit which is designed to locally modify the contour of the hollow organ representation on the basis of the command input, taking into consideration a predefined number of contour representations which are adjacent to the selected contour representation on at least one side along the hollow organ course line, using an automatic interpolating sweep algorithm.

The first provision unit may be realized as a simple receive interface for the image data and/or the hollow organ course line, but may also comprise an acquisition unit for acquiring the image data and/or a course line generator which, during operation, generates the hollow organ course line on the basis of the image data. Therefore the first provision unit may also comprise a plurality of subunits.

The second provision unit can also be combined with the first provision unit, e.g. as a subsidiary unit.

Overall, the creation and/or modification system described above is designed to carry out the method described above for interactively creating and/or modifying a hollow organ representation.

Overall, most of the components which together inventively form the creation and/or modification system, in particular the first and second provision unit, the receive interface and the modification unit, can be realized wholly or partially in the form of software modules on a processor. A plurality of the units can also be combined to form a general-purpose functional unit.

The (cited and optionally further) interfaces need not necessarily be designed as hardware components, but can also be realized as software modules, e.g. if the image data can be imported from another component which is already realized on the same device, e.g. an image reconstruction apparatus or similar, or need merely be transferred via software to another component. Likewise, the interfaces may consist of hardware and software components, e.g. a standard hardware interface which is individually configured by way of software for a specific purpose. Moreover, a plurality of interfaces can also be combined to form a general-purpose interface, e.g. an input/output interface.

At least one embodiment of the invention therefore also comprises a computer program product which can be loaded directly into a processor of a programmable creation and/or modification system, having program code segments for executing all the steps of a method according to at least one embodiment of invention (also in accordance with the aspects described below) when the program product is executed on the creation and/or modification system.

At least one embodiment of the invention further comprises a medical-technical recording system comprising a recording unit (i.e. an acquisition unit) and a creation and/or modification system according to at least one embodiment of the invention (also in accordance with the aspects described below).

Further particularly advantageous embodiments and developments of the invention are derived from the dependent claims and the following description. The creation and/or modification system can also be developed in accordance with the respective dependent method claims in this case, or vice versa.

The command input is preferably effected on the basis of a number of user inputs. This means that a user applies individual judgement to intervene via user inputs in the automatic function for generating the hollow organ representation, and that the command input is not (or is only partly) based on an inherent automatic function of the creation and/or modification system. “Interactive” therefore means controlled (at least partly) by user intervention.

So-called implicit indicator functions have a particular role to play in the context of the invention. They are preferably used here to define the contour representations. In other words, the contour representations preferably comprise implicit indicator functions, most preferably planar implicit indicator functions.

Implicit indicator functions have the advantage that they represent a geometric object, in particular a closed geometric object, by way of a formula which can be used in a very flexible way. According to an example (preferred) embodiment, the surface or boundary line of the object can be defined by the value 0 in this case, while values on this side and that side of the 0, i.e. negative and positive values, are then automatically assigned to the interior and the exterior according to the function. Instead of therefore specifying coordinates of the object, such a function is used to define it. Since it is fundamentally of importance in the context of the invention as a whole to identify the surface or boundary line and the interior of contour representations, particularly when performing the sweeping algorithm, the use of an implicit indicator function offers inter alia the advantage of ease of use and interpolation.

The method described above makes use of local sweeping on the basis of a predefined number of adjacent contour representations. In order to minimize the sweeping effort while nonetheless achieving sufficiently good interpolation results, this number is preferably no higher than 5, more preferably no higher than 3, and most preferably no higher than 2.

The hollow organ representation usually has a beginning and an end, each of which is defined by a terminating contour representation. In this context, provision is preferably made for the plurality of contour representations to comprise two terminating contour representations, at essentially opposite ends of the hollow organ representation in each case, and for neither or only one of the terminating contour representations to be considered and/or modified during the local modification in step d). In other words, provision is made for avoiding the inclusion of both terminating contour representations in the method described above, since otherwise the local character of the sweeping method is lost.

Two further aspects emerge in the context of at least one embodiment of the invention and are described in greater detail below. They also stand alone per se, but are preferably applied in the context of the method described above.

The first further aspect relates to a method for preferably automatic modification of a hollow organ course line of a hollow organ representation (wherein the hollow organ again comprises specifically a blood vessel or a blood vessel tree) on the basis of medical-technical image data of a hollow organ (of an organism), in particular in the context of the method according to at least one embodiment of the invention as described above for interactively creating and/or modifying a hollow organ representation. The method of at least one embodiment comprises at least the following steps:

i) providing the medical-technical image data together with the hollow organ course line representing (again at least approximately) the course of the hollow organ,
ii) providing a plurality of contour representations of a contour of the hollow organ representation along the hollow organ course line,
iii) determining a deviation of the hollow organ course line from contour centerpoints of two adjacent contour representations,
iv) adapting the hollow organ course line with the aid of the two contour centerpoints if a predetermined maximal tolerated deviation of the hollow organ course line from the two contour centerpoints is exceeded, and
v) optionally: locally modifying the contour of the hollow organ representation on the basis of the adapted hollow organ course line.

This first further aspect of at least one embodiment of the invention therefore relates to the correction of the hollow organ course line, in particular a centerline as described in greater detail above, which depicts the central course of the hollow organ. With respect to some regions, insufficient information is available in image data to fully depict a contour of a hollow organ, e.g. because the distance between generated contour representations of the hollow organ is too great in specific regions, and therefore deviations from the actual course of the hollow organ can occur when specifying the hollow organ course line. This applies in particular when the hollow organ exhibits significant curvature in a specific region. The hollow organ course line provides an inadequate depiction of the course of the hollow organ in this region, and this presents a problem if flows in the interior of the hollow organ are subsequently to be simulated or calculated on the basis of the hollow organ representation created using the hollow organ course line. In the event of excessive deviation (i.e. a deviation which is greater than the maximal tolerated deviation cited above) of the hollow organ course line from two adjacent contour centerpoints, the first further aspect described here uses these centerpoints as an orientation aid for adaptation of the hollow organ course line. The maximal tolerated deviation is usually exceeded if the relevant hollow organ exhibits a significant curvature between the two relevant adjacent contour representations. The corresponding adaptation of the hollow organ course line is used to depict this curvature instead of following the hollow organ course line which was previously calculated and is no longer sufficiently consistent here.

The adapted hollow organ course line can fully or (preferably) partly replace the original hollow organ course line in this context, or it can be used in addition to the original hollow organ course line in order to describe more effectively, i.e. more accurately, the course of the hollow organ in the hollow organ region defined by the two cited adjacent hollow organ representations.

The adaptation of the hollow organ course line is preferably performed in the tangent space, thereby simplifying the interpolation. As a result of the transformation into the tangent space, the adapted hollow organ course line is actually automatically aligned with the course of the original hollow organ course line.

The modification system corresponding to this first further aspect, for modifying a hollow organ course line of a hollow organ representation on the basis of medical-technical image data of a hollow organ, comprises:

i) a first provision unit which, during operation, provides the medical-technical image data together with the hollow organ course line representing (again at least approximately) the course of the hollow organ,
ii) a second provision unit which, during operation, provides a plurality of contour representations of a contour of the hollow organ representation along the hollow organ course line,
iii) a deviation determining unit which is designed to determine a deviation of the hollow organ course line from contour centerpoints of two adjacent contour representations,
iv) an adaptation unit which, during operation, adapts the hollow organ course line with the aid of the two contour centerpoints if a predetermined maximal tolerated deviation of the hollow organ course line from the two contour centerpoints is exceeded.

Overall, the modification system is designed to carry out at least one embodiment of the inventive method described above in accordance with the first further aspect.

Here likewise, most of the components which together inventively form the modification system, in particular the first and second provision unit, the deviation determining unit and the adaptation unit, can be realized wholly or partially in the form of software modules on a processor. A plurality of the units can also be combined to form a general-purpose functional unit.

Interfaces need not necessarily be designed as hardware components, but can also be realized as software modules, e.g. if the image data can be imported from another component which is already realized on the same device, e.g. an image reconstruction apparatus or similar, or need merely be transferred via software to another component. Likewise, the interfaces may consist of hardware and software components, e.g. a standard hardware interface which is individually configured by way of software for a specific purpose. Moreover, a plurality of interfaces can also be combined to form a general-purpose interface, e.g. an input/output interface.

In the context of the first further aspect, the hollow organ course line is preferably so adapted as to be routed essentially through the two contour centerpoints. The previously determined contour centerpoints are therefore used not only to recognize that the maximal tolerated deviation has been exceeded, but also subsequently to reorient the hollow organ course line in that the hollow organ course line is routed through them. This ensures that the hollow organ course line at the two centerpoints runs through the center of the hollow organ in each case, and therefore course deviations away from this center between the two contour representations are also as small as possible given the data that is available. The accuracy of the course of the hollow organ course line can thus be increased in an effective and targeted manner.

In order to achieve even greater accuracy and in particular to increase the smoothness of the course of the resulting hollow organ representation, additional location information relating to the further contour representations adjacent again to the two adjacent contour representations is preferably taken into consideration for the purpose of adapting the hollow organ course line. The hollow organ course line is therefore not readjusted solely on the basis of the location information of the two previously cited adjacent contour representations, but on the basis of the location information of further contour representations along the hollow organ course line on this side and/or that side (preferably both) of the two adjacent contour representations concerned.

The second further aspect relates to a method for semi-automatic modification of a contour representation of a hollow organ representation on the basis of medical-technical image data of a hollow organ, particularly in the context of step d) of the method described above for interactively creating and/or modifying a hollow organ representation. It comprises the steps:

I) providing the medical-technical image data together with a number of contour representations of a contour of the hollow organ representation,
II) receiving a command input for the input and/or modification of a selected contour representation from the contour representations,
III) adapting a geometric (virtual) object having a predefined shape into the image data at the location of the selected contour representation,
IV) merging geometric information from the command input from step II) and information relating to the geometric object from step III) to form a merged modified contour representation instead of the selected contour representation.

The adaptation in step III can also be referred to as incorporation of the geometric object. Such a geometric object is preferably a self-contained object and is used in the context of the adaptation step III as an ideal-typical mathematical description or definition of a contour of the hollow organ concerned. It therefore preferably depicts a contour and in particular a shape, e.g. a circle or an ellipsis, which is to be expected at essentially the position of the hollow organ. Completion of the step IV results in the modified contour representation, which is now introduced into the hollow organ representation instead of the previously selected contour representation. Any desired mixture variants between the ideal-typical description (based on the geometric object) and the contour-based description (based on the contour representation) are possible in this context. This allows an infinitely variable setting of the permitted deviation of the locally existing shape from the ideal-typical or expected shape. Accordingly, provision is most preferably made for performing local sweeping as per the invention (and explained in detail above) following this introduction/replacement.

The creation and/or modification system corresponding to said second further aspect, for interactively creating and/or modifying a hollow organ representation on the basis of medical-technical image data of a hollow organ, accordingly comprises:

I) a provision unit which, during operation, provides the medical-technical image data together with a number of contour representations of a contour of the hollow organ representation,
II) a receiving unit which is designed to receive a command input for the input and/or modification of a selected contour representation from the contour representations,
III) an incorporation unit which, during operation, incorporates a geometric object having a predefined shape into the image data at the location of the selected contour representation,
IV) a merging unit which, during operation, merges geometric information from the command input of the receiving unit and information relating to the geometric object from the incorporation unit to form a merged modified contour representation instead of the selected contour representation.

Overall, the creation and/or modification system is designed to perform the inventive method described above in accordance with the second further aspect.

Here likewise, most of the components which together inventively form the creation and/or modification system, in particular the provision unit, the receiving unit, the incorporation unit and the merging unit, can be realized wholly or partially in the form of software modules on a processor. A plurality of the units can also be combined to form a general-purpose functional unit.

Interfaces need not necessarily be designed as hardware components, but can also be realized as software modules, e.g. if the image data can be imported from another component which is already realized on the same device, e.g. an image reconstruction apparatus or similar, or need merely be transferred via software to another component. Likewise, the interfaces may consist of hardware and software components, e.g. a standard hardware interface which is individually configured by way of software for a specific purpose. Moreover, a plurality of interfaces can also be combined to form a general-purpose interface, e.g. an input/output interface.

In the context of the second further aspect likewise, the command input is preferably effected on the basis of a number of user inputs. The advantages are similar to those cited above.

The geometric object is preferably self-contained as described above, wherein its boundary line may also be angular in principle. It preferably comprises a circle or an ellipsis or an oval or a polygon, in which case it is most preferably a rotationally symmetrical polygon. These shapes can depict a hollow organ contour in a particularly clear and ideal-typical manner, and are therefore particularly suitable.

In this context likewise, the selected contour representation and/or the geometric object is preferably represented by an (in particular planar) implicit indicator function. The advantages cited above also apply to this preferred embodiment.

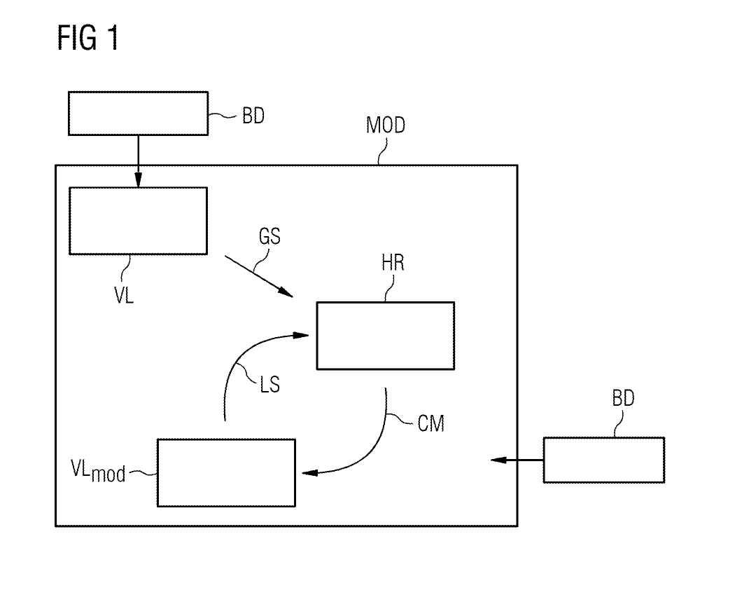

FIG. 1 shows a first schematic sequence diagram of a method for segmenting a hollow organ, in particular a blood vessel, wherein various aspects of the invention can be applied in the performance of said method. Medical-technical image data BD of the relevant hollow organ is provided by way of image acquisition and is used for geometric modeling MOD. For this purpose, a hollow organ course line VL, for example a centerline VL of the relevant hollow organ, is derived or generated from the medical-technical image data BD. In the context of global sweeping GS, provision is made for sweeping along the hollow organ course line VL on the basis of a mask or grid and a three-dimensional model HR, specifically a hollow organ representation HR, is generated therefrom. On the basis of the medical-technical image data BD, it is also possible in a contour modification step CM to create a modified hollow organ course line VL_{mod}, which e.g. corresponds more closely to the hollow organ course than the hollow organ course line VL provided initially. An inventive procedure in this connection is described with reference to FIG. 3 in particular.

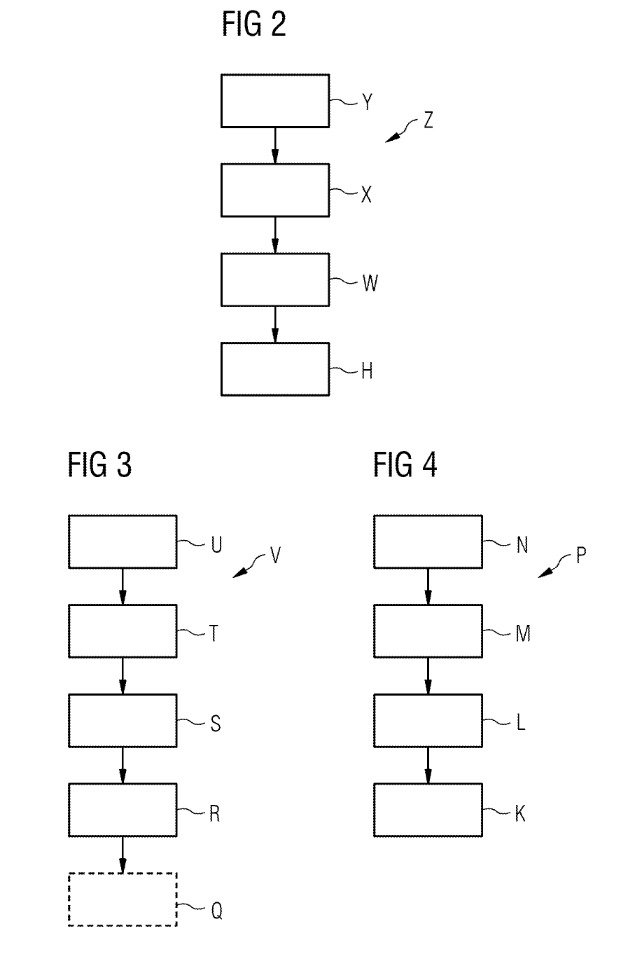

In addition to this, the hollow organ representation HR can be modified by way of local sweeping LS, e.g. along the modified hollow organ course line VL_{mod}. This local sweeping LS is described with reference to FIG. 2, for example. A specific modification strategy is derived in particular from the method which is described in greater detail with reference to FIG. 4.

FIGS. 2 to 4 show in abstract form the steps of a method according to an embodiment of the invention, or of two individual aspects, wherein said steps have already been enumerated in the same order above.

Using a block diagram for the purpose of illustration, FIG. 2 shows an example embodiment of a method Z for interactively creating and/or modifying a hollow organ representation HR on the basis of medical-technical image data BD of a hollow organ. In a first step Y, the medical-technical image data BD is provided together with a hollow organ course line VL representing the course of the hollow organ. In a second step X, a plurality of contour representations of a contour of the hollow organ representation HR along the hollow organ course line VL are also provided. In a third step W, a command input is received W for the input and/or modification of a selected contour representation and/or of the hollow organ course line VL. In a fourth step H, the contour of the hollow organ representation HR is then adapted on the basis of the command input, taking into consideration a predefined number of contour representations which are adjacent to the selected contour representation on at least one side along the hollow organ course line VL, using an automatic interpolating sweep algorithm. This step corresponds to the local sweeping LS from FIG. 1.

FIG. 3 shows a block diagram of an example embodiment of the first (additional) aspect of the invention, specifically a method V for preferably automatic modification CM of a hollow organ course line VL of a hollow organ representation HR on the basis of medical-technical image data BD of a hollow organ. It comprises steps as follows:

In a first step U, the medical-technical image data BD is provided together with the hollow organ course line VL representing the course of the hollow organ. In a second step T, a plurality of contour representations of a contour of the hollow organ representation HR along the hollow organ course line VL are provided. In a third step S following thereupon, a deviation of the hollow organ course line VL from contour centerpoints of two adjacent contour representations is determined S and, in a fourth step R, the hollow organ course line VL is adapted R with the aid of the two contour centerpoints if a predetermined maximal tolerated deviation of the hollow organ course line VL from the two contour centerpoints is exceeded.

FIG. 4 shows a block diagram of an example embodiment of the second (additional) aspect of the invention, specifically a method P for the semi-automatic modification of a contour representation of a hollow organ representation HR on the basis of medical-technical image data BD of a hollow organ. The method P comprises the following steps:

In a first step N, the medical-technical image data BD is received together with a number of contour representations of a contour of the hollow organ representation HR, and in a second step M a command input is received M for the input and/or modification of a selected contour representation from the contour representations. A third step L consists in adapting L a geometric object having a predefined shape to the image data BD at the location of the selected contour representation, and fourth step K consists in merging K geometric information from the command input and information relating to the geometric object to form a merged modified contour representation instead of the selected contour representation.

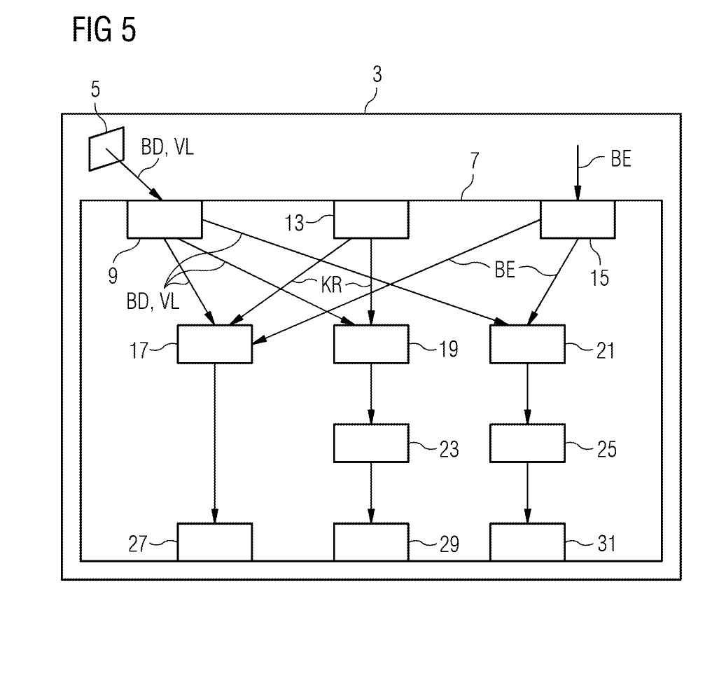

FIG. 5 shows a schematic block diagram of a medical-technical recording system 3 according to an embodiment of the invention, comprising a recording unit 5 and an embodiment of a creation and/or modification system 7 according to the invention.

The creation and/or modification system 7 is used here to perform all of the aspects of inventive methods Z, V, P, as described above with reference to the FIGS. 2 to 4. It has a first provision unit 9, a second provision unit 13 and a receive interface or receiving unit 15. It further comprises a modification unit 17, a deviation determining unit 19, an adaptation unit 23, an incorporation unit 21 and a merging unit 25. For the purpose of output, the creation and/or modification system 7 comprises three output interfaces 27, 29, 31.

The first provision unit 9 is designed as an input interface 9 here, and performs the step Y of the method Z or the step U of the method V or part of the step N of the method P. This means that it provides medical-technical image data BD of a hollow organ, together with a hollow organ course line VL representing the course of the hollow organ, in the first method Z and in the second method V.

The second provision unit 13 is used to perform the step X of the method Z or the step T of the method V or the second part of the step N of the method P. It is likewise designed as an input interface 13 and, during operation, provides a number, in particular a plurality, of contour representations KR of a contour of the hollow organ representation HR along the hollow organ course line VL.

The receive interface 15 or receiving unit 15 is designed to receive a command input BE for the input and/or modification of a selected contour representation (in the context of the step W of the method Z or the step M of the method P) and/or of the hollow organ course line VL (in the context of the step M of the method Z).

The modification unit 17 is used to perform the step H of the method Z and is therefore designed to locally modify the contour of the hollow organ representation HR on the basis of the command input BE, taking into consideration a predefined number of contour representations which are adjacent to the selected contour representation on at least one side along the hollow organ course line, using an automatic interpolating sweep algorithm. The modification unit 17 therefore performs local sweeping LS as described above.

The deviation determining unit 19 is used in conjunction with the adaptation unit 23 to perform the steps S and R of the method V: In this case, the deviation determining unit 19 determines a deviation of the hollow organ course line VL from contour centerpoints of two adjacent contour representations, and the adaptation unit 23 adapts the hollow organ course line VL with the aid of the two contour centerpoints if a predetermined maximal tolerated deviation of the hollow organ course line VL from the two contour centerpoints is exceeded.

The incorporation unit 21 and the merging unit 25 perform the steps L and K of the method P: In this case, the incorporation unit 21 incorporates a geometric object having a predefined shape into the image data BD at the location of the selected contour representation, and the merging unit 25 merges geometric information from the command input BE of the receiving unit 15 and information relating to the geometric object from the incorporation unit 21 to form a merged modified contour representation instead of the selected contour representation.

With the help of specific illustrative image data, formulas and cross-references to the extensive literature in the field of segmenting, an example embodiment of an execution of the method steps and method details according to the invention is explained in greater detail below with reference to particularly preferred embodiment details. References back and forth are occasionally made in this case by way of cross-references between the previously used terminology of the present application (in particular the claims) and specific mathematical technical terms in the example embodiment. For the sake of readability, these cross-references are however sporadic. It is assumed that the terminology of the example embodiment and the assigned terminology of the remainder of the application can generally be used synonymously unless explicitly stated otherwise.

Current vascular modeling methods can be categorized broadly into model-based methods and non-model methods.

Non-model methods are also referred to as implicit methods, since they are usually based on generic scatterplot interpolation techniques and use implicit indicator functions in order to interpolate models. These methods are normally concerned with the resilient extraction of scatterplots from binary segmentation masks, from which they can extract even fine vessels. In order to generate reliable interpolations, these methods require dense sampling and do not usually contain any explicit topological or geometric information relating to the underlying vascular structures.

By contrast, model-based methods are based on the tubular structure of vascular systems and are often used to visually depict course lines. Many of these techniques are based on explicit methods of mesh generation, which can normally be performed very quickly but also results in an overlapping of meshes at vascular branch points. For the purpose of computational hemodynamics, however, the underlying models must be smooth and free of self-overlapping or unwanted internal structures. The implicit modeling method provides inherent construction mechanisms in order to solve this problem and has already proven successful in the generation of model-based vascular models.

Implicit Modeling

Implicit indicator functions provide a compact formulation by which the volume and the surface of an object can be described in a scalar field d(x): |³→|. In the case of signed indicator functions, the surface of an object is usually defined by a zero level set, i.e. by d(x)=0, the interior of the object by d(x)<0 and the exterior of the object by d(x)>0.

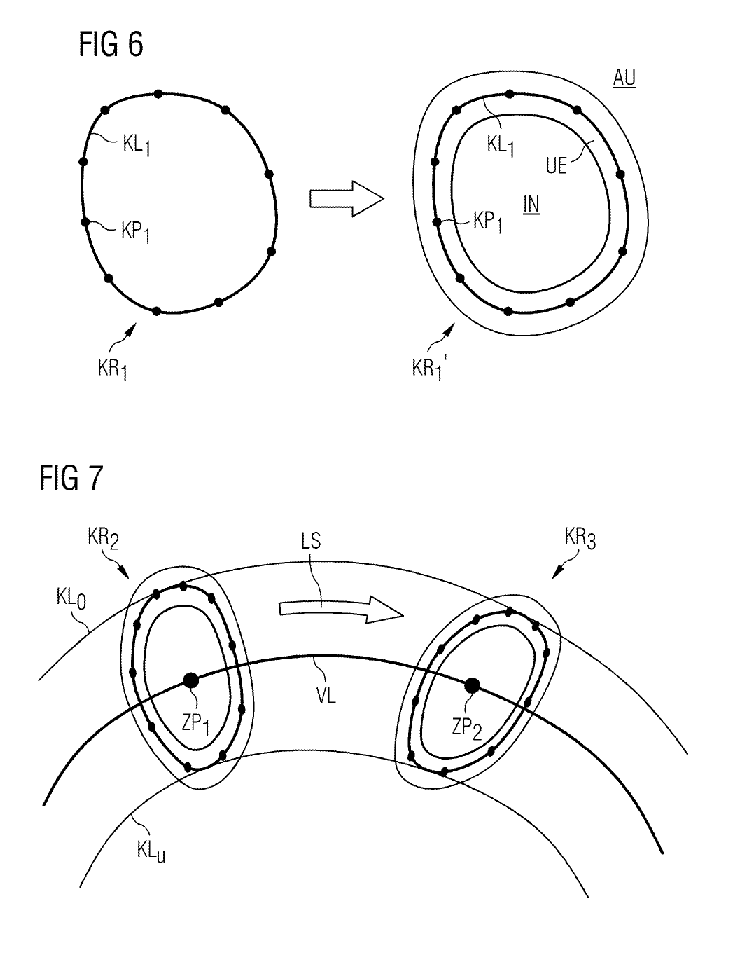

Specifically, FIG. 6 shows the initial scenario and the final scenario of such a process: a contour representation KR₁ having a number of contour points KP₁ on a contour line KL₁ is converted into a corresponding contour representation KR₁′, i.e. represented by an implicit function. The implicit function allows a distinction in the (right-hand) image between the exterior AU of the contour representation KR₁′ and the interior IN thereof, and additionally features an intermediate region UE in the center of which is situated the contour line KL₁ and the contour points KP₁ thereof. As mentioned above, the value of the implicit function can be defined as 0 for all contour points KP₁ on the contour line KL₁, such that the interior IN always has a negative function value of the implicit function and the exterior AU always has a positive function value of the implicit function.

Implicit modeling is based on the fact that signed indicator functions can easily be combined by using Boolean operators in order to define unions or intersections of sets. Such Boolean operators include e.g. min and max operators in this context. The result of combining two signed indicator functions is again a signed indicator function, which allows a recursive application of operators in order to form complex objects. This process is generally referred to as “solid modeling” and can easily be applied.

Sweep Objects

In the context of the invention, so-called sweep objects are defined as shapes which are produced when an object is moved along a path of travel. Depending on the application, solely the surface of the resulting sweep object (i.e. the sweep surface), or the volume thereof (i.e. the sweep volume), or both may be of interest. Accordingly, there are two fundamental associated fields of investigation: sweep volumes are concerned with the movement of three-dimensional objects within a space, while sweep surfaces are normally used to describe two-dimensional shapes which are moved, i.e. swept, along a path of travel.

The vascular cross-section information that is based on the vascular course line provides information about two-dimensional shapes (the contour representations) which are assigned to (a location of) the hollow organ course line VL. These two-dimensional shapes can be moved virtually by way of the sweep surface method.

FIG. 7 shows the basic course of a sweeping method with reference to two contour representations KR₂, KR₃. In this case, the first contour representation KR₂ is moved by way of local sweeping LS via interpolation along a hollow organ course line VL in a sweeping direction which is indicated by an arrow. The centerpoints ZP₁ of the first contour representation KR₂ and ZP₂ of the second contour representation KR₃ lie on the hollow organ course line VL, such that the hollow organ course line VL is a centerline VL. The local sweeping LS produces an exterior contour of the hollow organ as symbolized by the two contour lines KLₒ, KLᵤ.

Previous approaches of the sweeping method focused either on direct generation of resulting sweeps (i.e. sweep objects) in the specified parameter range or on direct visual depictions by way of beam tracking and applying analytical intersection tests. One significant problem in this case is that intersections can occur relatively frequently depending on the path of travel and the shape of the moving object, i.e. the moving shape template or the respective contour representation here. Implicit surface descriptions have the property that volumetric Boolean operations such as lattice or intersection formations can be performed easily. This property can be applied for the purpose of eliminating inherent self-intersections, specifically by using implicit functions as sweep templates, i.e. representing the moving objects (namely contour representations here) by way of implicit functions.

As described above, basic functions of the implicit sweeping method are defined by a sweep template and a path of travel along which the sweep template is moved. In the present case as shown in FIG. 7, the path of travel corresponds to the hollow organ course line VL of the vessel, more precisely of an unbranched vascular segment, wherein the hollow organ course line VL can be described by a number successive points which (in the case of a centerline) are located in the centerpoint of the vessel. The corresponding vascular contours, i.e. contour representations, are represented in this case by a number of coplanar points which lie in the corresponding plane on the surface of the vessel and define a closed polygon.

In order to create an implicit description of a vascular tree, three steps are performed: Firstly, all vascular contours, i.e. contour representations, are converted into implicit two-dimensional sweep templates. Next, the course line of the vessel is used to calculate the sweep surface of each individual vessel of a vascular tree. Finally, the individual vessels are smoothly blended (i.e. mixed) to form the complete vascular tree.

Implicit Sweep Templates

There are numerous methods for generating implicit descriptions of an object from a polygon. In order to allow interactive realtime applications, rasterized implicit contour representations are preferably calculated in advance. These previously calculated images can be used as implicit sweep templates during the evaluation of the subsequent implicit sweeping, such that distance calculations in the application area of the sweep templates involve nothing more than a simple and rapid image lookup, i.e. a sort of “cache” strategy. In order to avoid step effects, standard linear filtering methods can be used when accessing the sweep template images.

Catmull-Rom splines are a frequently used group of cubic Hermite interpolants (cf. in particular Tony D. DeRose/Brian A. Barsky: “Geometric continuity, shape parameters, and geometric constructions for Catmull-Rom splines” in: ACM Transactions on Graphics (TOG), volume 7, issue 1, January 1988, pages 1-41, doi:10.1145/42188.42265, the entire contents of which are hereby incorporated herein by reference). They are used here for a plurality of interpolation tasks, since they are easy to construct. An approximately curved and longitudinally parameterized Catmull-Rom spline which interpolates a continuous list of points pi, i=0 . . . n is defined as

$${CR}_{{p}_{i}}^{τ} ( t ) = {( \begin{matrix}1 \\ u \\ {u}^{2} \\ {u}^{3}\end{matrix} )}^{T} ( \begin{matrix}0 & 1 & 0 & 0 \\ - τ & 0 & τ & 0 \\ 2 τ & τ - 3 & 3 - 2 τ & - τ \\ - τ & 2 - τ & τ - 2 & τ\end{matrix} ) ( \begin{matrix}{p}_{i} - 1 \\ {p}_{i} \\ {p}_{i} \\ {p}_{i} + 2\end{matrix} )$$

$$u = \frac{t - {t}_{i}}{{t}_{i + 1} - {t}_{i}}$$

where tᵢ=|pᵢ−pᵢ₋₁| and t₀=0 identifies the approximation of the curve length. For each interval [tᵢ, tᵢ₊₁], the spline is defined by a local Bezier curve which starts at pᵢ and ends at pᵢ₊₁. The user-adjustable parameter value τε[0, 1] identifies the so-called “tension”, which therefore signifies the degree of spline “curvature” in this region.

In order to generate shape templates (i.e. sweep templates) efficiently, provision is made in a first step for constructing a closed Catmull-Rom spline CR_{O}_{ᵢ}^{0,7} which interpolates contour representations. In order to convert this parameterized curve into an implicit description, the spline is adaptively subdivided and the resulting polygon is rasterized to produce an image of adaptable resolution. This is followed by the use of an unsigned distance transformation algorithm which results in a discrete distance field. In order to convert this into a signed distance field, a modified scan line algorithm is applied which uses the subdivided polygon and reverses the operational sign of the distance values within the object.

This method is very fast, and the quality of the resulting distance fields is determined primarily by the selection of the distance transformation algorithm and by the raster resolution. The discrete distance templates generated using this approach are externally truncated beyond a certain distance from the contour, but since the main interest of the invention is directed at the surface and the interior of the sweep object that is ultimately produced, those zones of the distance field which are more remote from the object are of little interest in any case.

In order to increase the performance, the templates can be distributed over available computing units for serial-parallel generation.

Definition of an Implicit Sweep Object

In a next step, an implicit indicator function B(P) is calculated from a path of travel and assigned sweep templates as defined by the hollow organ course line VL. The surface of a vascular branch can be described by the zero level set B(P)=0, the interior of the vessel as negative zones where B(P)<0.

F: |→|³ is a smooth parametric curve, which interpolates sampled course line positions Fi using parameter values ti. In this case, it applies that:

F(t)=(fₓ(t),f_{y}(t),f_{z}(t))

In order to define the sweep surface of the vessel, an affine mapping W: |→|³ is required, which transforms positions from the parameter field of the curve into the enclosed space and vice versa. In the following, the parameter field is referred to as tangent space and the enclosed space as world space. In this case, the tangent space can be defined using Frenet frames which define an orthonormal basis for each curve parameter t:

$${F}^{t} ( t ) = \frac{{F}^{′} ( t )}{‖ {F}^{′} ( t ) ‖}$$

$${F}^{n} ( t ) = \frac{{F}^{″} ( t )}{‖ {F}^{″} ( t ) ‖}$$

$${F}^{b} ( t ) = {F}^{t} ( t ) \times {F}^{n} ( t )$$

where F^{t} is the tangent function, and the normal function Fⁿ and the binormal function F^{b} parameterize a plane which is always orthogonal to the hollow organ course line VL. The basis defined by F^{t}, Fⁿ and F^{b} makes it possible to define the mapping W, which transforms tangent space points P′=(Pₜ, Pₙ, P_{b}) into world space points P=(Pₓ, P_{y}, P_{z}) for each specified curve parameter t.

(Pₓ,P_{y},P_{z})=Wₜ(Pₜ,Pₙ,P_{b})=F(Pₜ)+Pₙ*Fⁿ(Pₜ)+P_{b}*F(Pₜ)

In order to describe curved hollow organ course lines VL, F(t) can be defined using a Catmull-Rom spline CR_{O}_{ᵢ}^{0,7}(t) which interpolates the sampled course line positions Fi. Each world space position can be mapped onto a plurality of positions in the tangent space. This initially means that local self-overlaps can occur depending on the curvature of the path of travel (i.e. the hollow organ course line VL) and on the extent of the sweep templates used. As a result of the implicit definition when sweeping, these self-overlaps can however easily be resolved by applying a Boolean lattice formation operator.

In order to specify W⁻¹ for a point P in the world space, it is necessary to specify all of the parameter values t which allow a projection of P onto F(t) in the plane which is defined by F(t). The opposite mapping W⁻¹ therefore appears as follows:

W⁻¹(Pₓ,P_{y},P_{z})={Wₜ⁻¹(Pₓ,P_{y},P_{z})|(Pₓ,P_{y},P_{z})−F(t))·F^{t}(t)=0}

The calculation of the projectable parameters t is dependent on the degrees of the polynomials which were used to describe F and Fⁿ. In the case of cubic polynomial interpolations, which is the preferred case here, the zeroes of a polynomial of the fifth degree must be calculated. This root identification is performed individually for each local Bezier curve segment of the relevant Catmull-Rom spline. As soon as the curve parameters t for a specific point (Pₓ, P_{y}, P_{z}) in the world space have been determined, this point can be mapped onto all corresponding positions in the tangent space, the implicit sweeping can be evaluated, and the implicit values can be derived by way of the lattice formation operator.

B(Pₓ,P_{y},P_{z})=minₗᵢ{B′(W⁻¹(Pₓ,P_{y},P_{z})}

Since sweep templates are often few in the case of certain curve parameters t, an interpolation method may be required which conducts the sweeping via intermediate positions. In the case of tangent space points, the Pₜ coordinate corresponds directly to the curve parameter t, resulting in the generation of a batch of sweep templates.

This configuration allows direct interpolation between sweep templates. In order to blend the distance field templates, use is made of the Catmull-Rom CR_{O}_{ᵢ}^{0,7} splines cited above, wherein Tᵢ=Tᵢ(Pₙ, P_{b}) are the values, sampled from the implicit sweep template, which are linked to the parameter value tᵢ. The implicit distance value for a point P=(Pₜ, Pₙ, P_{b}) in the tangent space is therefore defined as follows:

B′(Pₜ,Pₙ,P_{b})=CR_{O}_{ᵢ}^{0,7}(Pₜ)

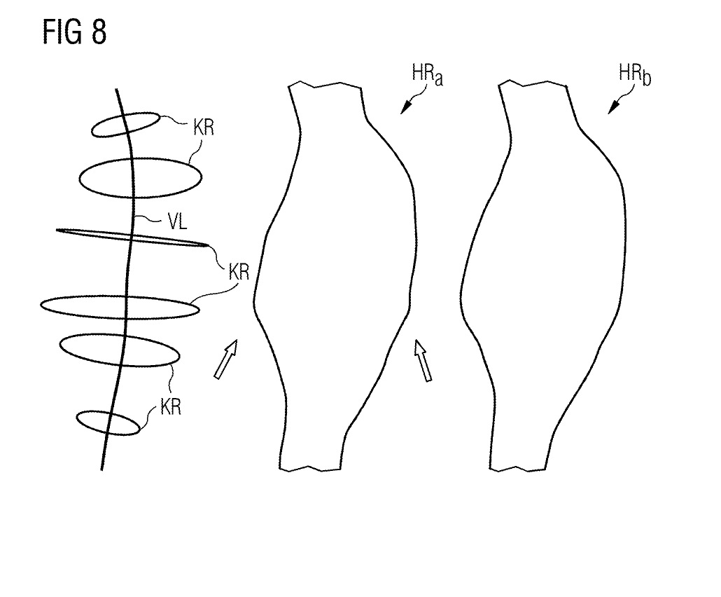

FIG. 8 shows a comparison between sweeping results of a linear sweep interpolation (center) and an interpolation on the basis of a Catmull-Rom spline (right). Starting from a number of known contour representations KR along a hollow organ course line VL (left), a first hollow organ representation HRₐ was generated on the basis of the first-cited method and a second hollow organ representation HR_{b} on the basis of the second-cited method. While the first hollow organ representation HRₐ has corners, as indicated by arrows, which portray the underlying organic structure only poorly, the second hollow organ representation HRₐ is considerably smoother and therefore also far better represents the hollow organ. This FIG therefore underlines the improvement of the modeling quality when Catmull-Rom splines are used.

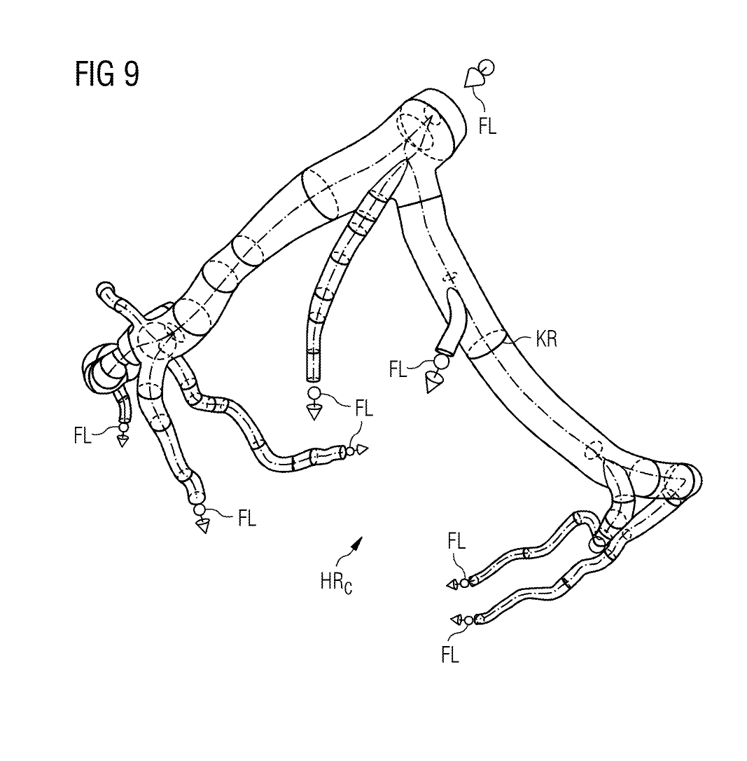

FIG. 9 shows a complete vascular tree of a blood vessel structure in the form of a hollow organ representation HR_{c} such as can be portrayed using the modeling algorithms described above. In addition to the spatial distribution of the vascular tree, also identifiable and indicated is the flow direction FL in which the blood flows into the system and then onward.

Resilient Template Blending

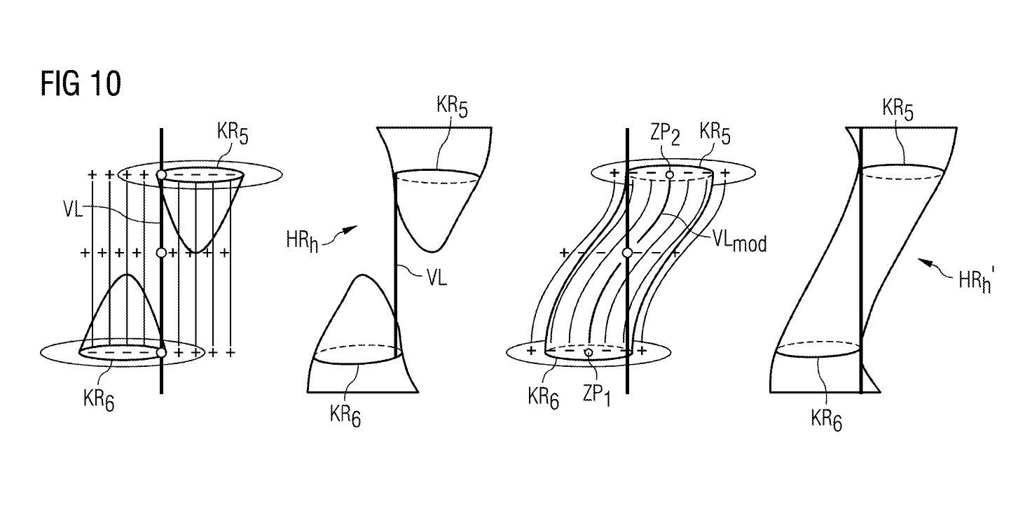

Interpolations of signed distance fields must be handled with care generally. For example, problems occur if the (negative) internal region of adjacent sweep templates does not lie along a line. This can even result in e.g. the extreme case in which the respective interiors of two adjacent sweep templates do not overlap each other at all, as shown in FIG. 10.

If the distance fields are then interpolated during the sweep process, the resulting volume, i.e. the resulting hollow organ representation, has two zones which are separated from each other (left-hand side of FIG. 10, right-hand illustration) due to the existence between them of regions which all have a positive value (cf. left-hand side, left-hand illustration) as a result of the implicit functions of both sweep templates, i.e. the adjacent contour representations KR6 (below) and KR5 (above) in this case. It can also be seen that the hollow organ course line VL does not run centrally through the two contour representations KR₅, KR₆ in each case. As a result of the (in this case lateral) offset of the two contour representations KR₅, KR₆, the hollow organ course line VL runs exactly through their respective boundary lines in this particular case.

If the interiors of both adjacent sweep templates overlap at least partially, the volume can normally be connected by corresponding sweep algorithms since the interpolated sweep templates contain negative interconnectable zones. However, there is also a danger here that these negative interconnectable zones of only slight overlap may have such small dimensions that volume artifacts can occur, and these could then be incorrectly interpreted as stenoses. In the case of well-determined hollow organ course lines VL, in particular well-defined centerlines as hollow organ course lines VL, this specific problem should not occur. It can be assumed in such cases that the centerline actually always runs through the center of the vessel and therefore prevents any such artifacts. In pathological cases, in the case of significant vascular calcification or in the case of noisy volumetric image data records having low resolution, automatic segmentation algorithms often produce inconsistent vascular models, in which only a limited number of the vascular contours (i.e. also the contour representations) are present and moreover the centerlines are not always sufficiently precise.

In order to ensure that such configurations inherently produce a satisfactory result, a spline-based blending technique is used in the context of the invention (this relates in particular to the first above-cited additional aspect of the invention), wherein said technique interconnects the sweep templates (preferably in the tangent space). Blending does not take place along the hollow organ course line VL as shown on the left-hand side of FIG. 10, but along an additional Catmull-Rom spline as illustrated on the right-hand side of FIG. 10. This spline is so constructed as to run through the contour centerpoints ZP₁, ZP₂ of the two adjacent contour representations KR₅, KR₆ (preferably in the tangent space). This technique significantly increases the resilience of model formation in the cases cited above.

Concerning the details of the procedure, reference is again made to the right-hand side of the FIG. 10. A corresponding contour centerpoint ZP₁, ZP₂ is determined for each of the adjacent contour representations KR₅, KR₆, said centerpoint preferably being the core of the respective contour representation KR₅, KR₆. If the hollow organ course line VL deviates from at least one of the contour centerpoints ZP₁, ZP₂ to the extent that the deviation value is greater than a maximal tolerated deviation, the hollow organ course line VL is so modified as to produce a modified hollow organ course line VL_{mod} which runs through the two contour centerpoints ZP₁, ZP₂ here. This results in a hollow organ representation HRₕ, which is modified in comparison with the originally generated hollow organ representation HRₕ.

Connection (Blending) of Vascular Branches

In order to model whole vascular trees, the previously constructed individual implicit branches must be consolidated to form a global implicit indicator function. In order to ensure an assembly having smooth transitions at branch points in this case, an extension of the so-called Wyvill field function is used:

F_{wyvill}(x)=(1−x²)³:|→[0,1]

A pseudo distance field of an individual vascular branch is therefore mapped relative to a potential field. In contrast with the forms cited above, the field function f_{w}(x): |→[−1,0] used here generates fields which are negative within objects (i.e. the contour representations). This means that min operators can be used consistently in order to form unified volumes:

$${f}_{w} ( x ) = \{ \begin{matrix}- 1 & x ≺ - w \\ - {f}_{wyvill} ( \frac{x}{2 w} + {f}_{wyvill}^{- 1} ( 0.5 ) … & - w \leq x \leq w \\ 0 & x ≻ w\end{matrix}$$

In this formula, w makes it possible to adapt how fast f_{w} falls to −1 within the object and to 0 outside of the object. This means that w represents the blending region of the described field. While distance fields generally deliver values unequal to zero for points at any distance in principle, and the object surface is defined by the zero level set, f_{w} is only unequal to zero in the vicinity of and within the object and the surface is defined by the level set −0.5.

An extensive selection of blending operators can be used in order to achieve smooth transitions between fields.

Interactive Model Correction

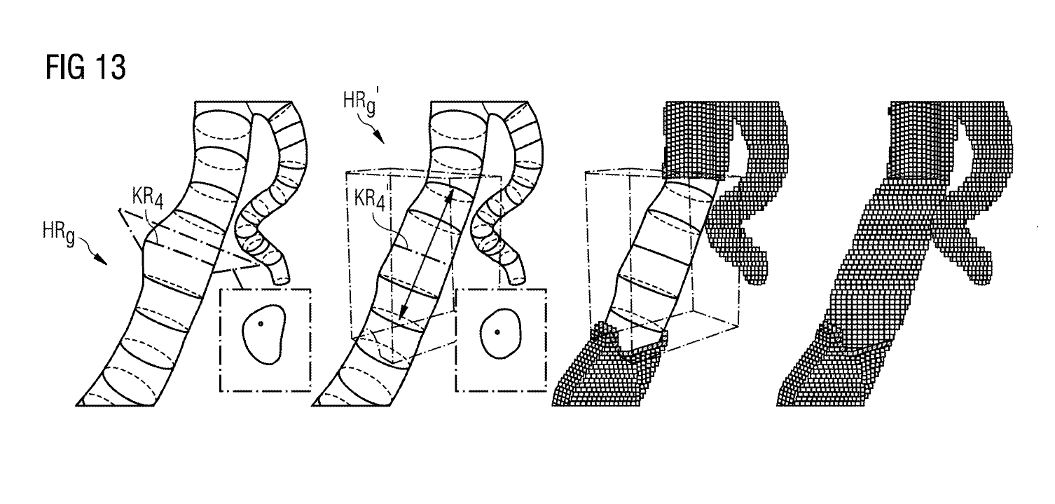

The example interaction framework presented here for model correction, i.e. for modifying a hollow organ representation, uses e.g. triangular meshes to visually depict a current vascular model. In addition, a volume data set can be loaded in order to synchronize and/or verify the segmenting in superimposed renderings. For example, computed tomography angiography image data offers a contrast medium-based view of patient-specific vascular structures, and therefore such image data is particularly suitable for vascular modeling. When a user clicks on any desired point of a three-dimensional mesh in such prepared image data, an associated MPR view (multiplanar reformation) is displayed. The user can then see the local vascular cross-section in the current vascular model as an overlay over the original image. This is shown in the first illustration (from the left) in FIG. 13. Using the mouse wheel, the user can navigate up and down through the relevant vascular branch (along the vascular cross-sections), in order to obtain a rapid overview of the current vascular model. A so-called “snapping feature” can also be activated if desired, whereby the respective view automatically adjusts itself such that the image includes the most obvious contour of the respective blood vessel.

If the user is dissatisfied with the vascular model at a specific position, he can draw a corrected contour into the MPR view in order for a modified complete vascular model to then be presented by way of the rapid local sweeping method according to the invention. If a contour representation was already present in this case, it is replaced, otherwise a new contour representation is inserted, and if a contour representation is erased by the user, it is deleted from the vascular model.

By way of example, FIG. 12 shows such a local sweeping LS in the image. The (original) hollow organ representation HRₑ of a hollow organ 1 comprises a multiplicity of contour representations KR_{anf}, KRₐ, KR_{b}, KR_{c}, KR_{d}, KR₄, KRₑ, KR_{f}, KR_{g}, KRₕ, KR_{end}. In this case, terminating contour representations KR_{anf}, KR_{end} are situated at both ends of the hollow organ representation HRₑ. In the present case, in which a selected contour representation KR₄ is modified, namely replaced by a modified contour representation KR₄′ (here: narrowed), the local sweeping (cf. right-hand side in the FIG) merely results in a slight narrowing in the two immediately adjacent contour representations, but not beyond this. It is in any case a principle of the local sweeping that both terminating contour representations KR_{anf}, KR_{end} are never included and/or modified in the sweeping.

Segment-Based Culling

In order to generate a mesh for the visual depiction, implicit polygonizers such as e.g. marching cubes-based algorithms can be used in order to evaluate the implicit sweep V at (many) world space positions P=(Pₓ, P_{y}, P_{z}). Provision is preferably made for calculating all potential projections of hollow organ course lines VL at each evaluation of V, as described above. This step is very costly in computing terms, since all roots of a fifth-degree polynomial must be found in this case, as described above. In order to localize the calculations, a so-called culling strategy can be used which is based on axis aligned bounding boxes (AABBs). To this end, provision is preferably made for the Catmull-Rom spline of the hollow organ course line VL to be sectionally subdivided into its individual cubic Bezier curve segments, for which AABBs are then calculated. Since a priori only limited knowledge is available in respect of the existing surface of an individual Bezier segment, conservative estimates are heuristically specified for each segment as follows.

On the basis of the convex hull properties of Bezier curves, the local control polygon (i.e. the local contour representation) can be used to delimit the segment of the hollow organ course line. It is also possible to specify a maximal distance from a hollow organ course line position to a corresponding vascular contour.

An AABB, which therefore encompasses all of the local contour points and also encompasses for each of the contour points a region that is at least as large in each direction as the maximal vascular radius present, can be used as a reliable estimate for the maximal extent of the local segment.

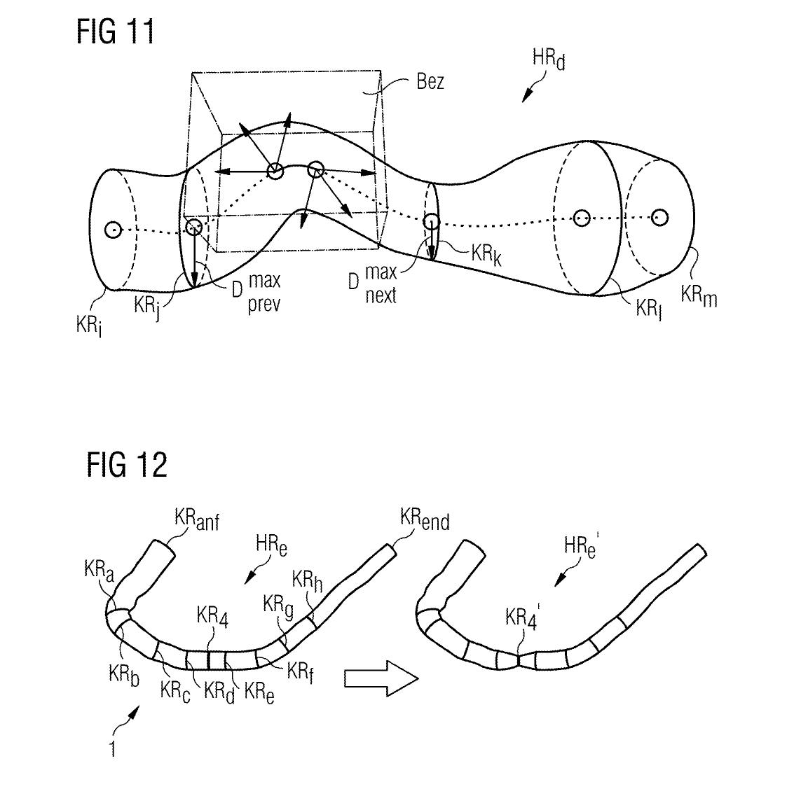

FIG. 11 shows the specification of the boundaries of a Bezier segment Bez. The AABB of its control points is calculated and extended in this case, until it has at most the maximal distances i.e. radii Dₚᵣₑᵥ^{max}, Dₙₑₓₜ^{max} of two adjacent contour representations KRⱼ, KRₖ from a series of contour representations KRᵢ, KRⱼ, KRₖ, KRₗ, KRₘ. Since the points on the hollow organ course line are not necessarily all associated with a contour representation, Dₚᵣₑᵥ^{max} and Dₙₑₓₜ^{max} here relate to the closest or adjacent known (i.e. non-empty) contour representations and their respective centerpoints on the hollow organ course line in each case. The AABBs thus calculated describe a worst-case approximation for the boundaries of a Bezier segment. Since the local cubic hollow organ course line curves also allow very complex configurations to be taken into consideration, this conservative but reliable boundary specifying technique was selected in the example. Since the chosen blending operator increases the influence of local segments, the AABBs might even require a further enlarged distribution which is proportional to the blending weighting. In order to allow rapid local queries in the image data, it is lastly possible to calculate an octree for the segment AABBs, as shown by both of the right-hand illustrations in FIG. 13. During a re-evaluation of the vascular model, this octree can be used to combine a local subset of hollow organ course line segments which must be taken into consideration when a point in the vascular model is changed.

Surface Extraction

In order to generate visual depictions effectively during an interactive session, a distribution-based marching cubes algorithm can be applied which uses mesh approximations of the supporting model.

Local Model Modifications

During a user interaction to modify the vascular model, the objective is to generate visual depictions of the vascular model which are updated a quickly as possible. This is the background to the procedure when sweeping as cited above. Therefore when a user deletes, changes or adds a new contour representation, the modification is limited locally to the previously generated AABBs concerned, based on the interpolation method and with reference to the Catmull-Rom spline. When an implicit template changes, two adjacent segments can potentially change their appearance. The vascular model may grow or become smaller during a modification. An AABB is therefore created which delimits the relevant segments and the model description with the new contour representation is then inserted. A further AABB is then created for the relevant segments and the two AABBs are combined such that new modified boundaries are produced. These steps are shown by the two left-hand illustrations in FIG. 13. In order then to create an updated surface mesh, it is sufficient to recalculate the surface exclusively within the new modified boundaries. For this purpose, the previous evaluations of the global distance function lying within the modified boundaries in the marching cubes volume are firstly deleted. All of the marching cubes cells currently cut by the surface and lying directly outside the modified boundaries are marked in this case. These cells are ideal entry points for a fresh calculation of the changed region (within the modified boundaries). Since it is guaranteed that they will still pass through the surface of the hollow organ after the change, they can be directly used as a starting point for a local recalculation by way of a distribution-based marching cubes method.

FIG. 13 can also be used to describe how a (e.g. user-defined yet also automated) modification of contour representations can proceed according to the second additional aspect of the invention. A selected contour representation KR₄ of the relevant hollow organ representation HR_{g} has been modified on the basis of a (computer-based and/or preferably user command-based) command input BE, as shown in the illustration on the far left. In addition to the command input BE, a geometric object having a predefined shape is used and is merged with the geometric information of the changed contour representation KR₄, thereby producing a merged modified contour representation KR₄′ (second illustration from the left) instead of the selected contour representation KR₄, said modified contour representation KR₄′ being used subsequently. It is clearly evident in the second illustration from the left that the incorporation and subsequent merging of the geometric FIG—here a circle that very well depicts the cross-section of the hollow organ which is essentially annular in this region—produces an improved approximation of the hollow organ shape that is actually to be expected.

Test Results

Tests using the modeling and/or model modification according to the invention revealed the following: While the complete initial model is generated in approximately 2.5 to 4.5 seconds in the sweeping method, a local modification using local sweeping in accordance with the invention takes less than 0.5 seconds, and is therefore fast enough to be displayed almost instantaneously to a user.

In conclusion, it is noted again that the method described in detail above and the apparatuses illustrated herein are merely example embodiments which may be modified in the widest variety of ways by a person skilled in the art without thereby departing from the scope of the invention. Moreover, use of the indefinite article “a” or “an” does not exclude multiple occurrence of the features concerned.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for interactively at least one of creating and modifying a hollow organ representation on the basis of medical-technical image data of a hollow organ, the method comprising:
providing the medical-technical image data together with a hollow organ course line representing a course of the hollow organ;
providing a plurality of contour representations of a contour of the hollow organ representation along the hollow organ course line;
receiving a command input for at least one of the input and modification of at least one of a selected contour representation and the hollow organ course line; and
locally modifying the contour of the hollow organ representation on the basis of the command input, taking into consideration a number of contour representations which are adjacent to the selected contour representation on at least one side along the hollow organ course line, using an automatic interpolating sweep algorithm.

2. The method of claim 1, wherein the command input is effected on the basis of a number of user inputs.

3. The method of claim 1, wherein the contour representations comprise implicit indicator functions.

4. The method of claim 1, wherein the number of adjacent contour representations is no higher than 5.

5. The method of claim 1, wherein the plurality of contour representations comprises two terminating contour representations, at essentially opposite ends of the hollow organ representation in each case, and wherein neither or only one of the terminating contour representations is at least one of considered and modified.

6. A method for automatically modifying a hollow organ course line of a hollow organ representation on the basis of medical-technical image data of a hollow organ, the method comprising:
providing the medical-technical image data together with the hollow organ course line representing a course of the hollow organ;
providing a plurality of contour representations of a contour of the hollow organ representation along the hollow organ course line;
determining a deviation of the hollow organ course line from contour centerpoints of two adjacent contour representations; and
adapting the hollow organ course line with aid of the two contour centerpoints upon a maximal tolerated deviation of the hollow organ course line from the two contour centerpoints being exceeded.

7. The method of claim 6, wherein the hollow organ course line is so adapted as to be routed essentially through the two contour centerpoints.

8. The method of claim 6, wherein additional location information relating to the further contour representations adjacent again to the two adjacent contour representations is taken into consideration for the purpose of adapting the hollow organ course line.

9. A method for semi-automatically modifying a contour representation of a hollow organ representation on the basis of medical-technical image data of a hollow organ, the method comprising:
providing the medical-technical image data together with a number of contour representations of a contour of the hollow organ representation;
receiving a command input for at least one of input and modification of a selected contour representation from the contour representations;
adapting a geometric object including a defined shape into the image data at a location of the selected contour representation; and
merging geometric information from the received command input and information relating to the adapted geometric object to form a merged modified contour representation instead of the selected contour representation.

10. The method of claim 9, wherein the command input is effected on the basis of a number of user inputs.

11. The method of claim 9, wherein the geometric object is a circle or an ellipsis or an oval or a polygon.

12. The method of claim 9, wherein at least one of the selected contour representation and the adapted geometric object is represented by an implicit indicator function.

13. At least one of a creation and modification system for interactively at least one of creating and modifying at least one of a hollow organ representation and a hollow organ course line of a hollow organ representation on the basis of medical-technical image data of a hollow organ, the system comprising at least one of:
a first provision unit configured to, during operation, provide the medical-technical image data together with a hollow organ course line representing a course of the hollow organ,
a second provision unit configured to, during operation, provide a plurality of contour representations of a contour of the hollow organ representation along the hollow organ course line,
a receive interface configured to receive a command input for at least one of input and modification of a selected at least one of contour representation and of the hollow organ course line, and
a modification unit designed to locally modify the contour of the hollow organ representation on the basis of the command input, taking into consideration a number of contour representations which are adjacent to the selected contour representation on at least one side along the hollow organ course line, using an automatic interpolating sweep algorithm; and/or
a first provision unit configured to, during operation, provide the medical-technical image data together with the hollow organ course line representing the course of the hollow organ,
a second provision unit configured to, during operation, provide a plurality of contour representations of a contour of the hollow organ representation along the hollow organ course line,
a deviation determining unit designed to determine a deviation of the hollow organ course line from contour centerpoints of two adjacent contour representations, and
an adaptation unit configured to, during operation, adapt the hollow organ course line with the aid of the two contour centerpoints upon a maximal tolerated deviation of the hollow organ course line from the two contour centerpoints being exceeded; and/or
a provision unit configured to, during operation, provide the medical-technical image data together with a number of contour representations of a contour of the hollow organ representation,
a receiving unit designed to receive a command input for the at least one of input and modification of a selected contour representation from the contour representations,
an incorporation unit configured to, during operation, incorporate a geometric object having a defined shape into the image data at the location of the selected contour representation, and
a merging unit configured to, during operation, merge geometric information from the command input of the receiving unit and information relating to the geometric object from the incorporation unit to form a merged modified contour representation instead of the selected contour representation.

14. A medical-technical recording system comprising:
a recording unit; and
the at least one of creation and modification system of claim 13.

15. A computer program product, directly loadable onto a processor of a programmable at least one of creation and modification system, including program code segments for executing the method of claim 1 when the program product is executed on the at least one of creation and modification system.

16. The method of claim 2, wherein the contour representations comprise implicit indicator functions.

17. The method of claim 1, wherein the number of adjacent contour representations is no higher than 2.

18. A method for automatically modifying a hollow organ course line of a hollow organ representation on the basis of medical-technical image data of a hollow organ, in the context of a method for interactively at least one of creating and modifying a hollow organ representation of claim 1, the method comprising:
providing the medical-technical image data together with the hollow organ course line representing a course of the hollow organ;
providing a plurality of contour representations of a contour of the hollow organ representation along the hollow organ course line;
determining a deviation of the hollow organ course line from contour centerpoints of two adjacent contour representations; and
adapting the hollow organ course line with aid of the two contour centerpoints upon a maximal tolerated deviation of the hollow organ course line from the two contour centerpoints being exceeded.

19. The method of claim 7, wherein additional location information relating to the further contour representations adjacent again to the two adjacent contour representations is taken into consideration for the purpose of adapting the hollow organ course line.

20. A method for semi-automatically modifying a contour representation of a hollow organ representation on the basis of medical-technical image data of a hollow organ, in the context of the step of the method for interactively at least one of creating and modifying a hollow organ representation of claim 1, the method comprising:
providing the medical-technical image data together with a number of contour representations of a contour of the hollow organ representation;
receiving a command input for at least one of input and modification of a selected contour representation from the contour representations;
adapting a geometric object including a defined shape into the image data at a location of the selected contour representation; and
merging geometric information from the received command input and information relating to the adapted geometric object to form a merged modified contour representation instead of the selected contour representation.

21. A computer program product, directly loadable onto a processor of a programmable at least one of creation and modification system, including program code segments for executing the method of claim 6 when the program product is executed on the at least one of creation and modification system.

22. A computer program product, directly loadable onto a processor of a programmable at least one of creation and modification system, including program code segments for executing the method of claim 9 when the program product is executed on the at least one of creation and modification system.

23. A computer program product, directly loadable onto a processor of a programmable at least one of creation and modification system, including program code segments for executing the method of claim 19 when the program product is executed on the at least one of creation and modification system.

24. A computer program product, directly loadable onto a processor of a programmable at least one of creation and modification system, including program code segments for executing the method of claim 20 when the program product is executed on the at least one of creation and modification system.
